(19) United States
(12) Patent Application Publication
(10) Pub. No.: US 2007/0258854 A1
(43) Pub. Date: Nov. 8, 2007

(54) Method of application of sterilization solution

(76) Inventor: James D. Fowle, St. Charles, MO (US)

Correspondence Address: Henry W. Cummings, 3313 W. Adams St., St. Charles, MO, 63301, US

(21) Appl. No.: 11/429,626

(22) Filed: May 8, 2006

(51) Int. Cl. A61L 2/22; A61L 2/20

(52) U.S. Cl. 422/28

(57) ABSTRACT

A method of application of a sterilization solution in laboratories, bio-safety cabinets, and other environments comprising delivering a hydrogen peroxide solution of preferably 3-29 percent hydrogen peroxide in sterilized water to the surface to be protected.

### I BACKGROUND OF THE INVENTION

Closest prior technique used to meet FDA Aseptic Sterilization Standards for laboratories, Bio Safety Cabinets, and other Critical Environments:
1) Hand application of sterilization solution by wiping all accessible surfaces. Negative aspect: Solution has to saturate organism for a sustained time. Evaporation makes this almost impossible.
2) Automated system VHP, pumps sterilization vapor into contaminated area of laboratory, Bio Safety Cabinet, and other Critical Environments. Negative aspects of this procedure are that contaminated areas must be sealed and pressurized with sterilization solution. Due to the difficulty in attaining an airtight seal, health and safety is a factor, with surrounding areas frequently evacuated.

### II SUMMARY OF PURPOSE OF THE INVENTION

A method of application which delivers sterilization solution in the form of an atomized spray or fog. Using this method with the correct solution and duration of delivery, will render a laboratory, Bio Safety Cabinet, and other Critical Environments Aseptically Sterile, meeting or exceeding FDA standards.

A) 3%-29% Hydrogen Peroxide in sterilized water has proven in my experiments and documentation, to achieve FDA Aseptic Sterilization Standards. 3% Hydrogen Peroxide in particular, is the invention's solution and can be increased as organisms become resistant. In addition, other solutions can be used to attain Aseptic Sterilization.

### III ADVANTAGE OF INVENTION

1) All surfaces can be selectively saturated by sterilization solution until target organism is eradicated.
2) Method of application will cover any area with sterilization solution for the proscribed duration needed to eradicate target organism.
3) No need to seal environment, because the space is not pressurized. Technician is delivering product at low pressure and low volume, directly to contaminated areas.
4) Due to the flexibility of directing the sterilization solution to specific areas for a sustained duration, for the first time, a 3% Hydrogen Peroxide solution has been used to attain Aseptic Sterilization, in accordance with FDA standards in a laboratory 80 ft.×50 ft.×12 ft., that was saturated with Aspergillus Phoenicia, Penicillium Olsonii, Kuoria, Streptococcus Salivarins, and Staphylococcus Epidermidis (description is attached).

A) The previous method of application used 30% or greater Hydrogen Peroxide, which is highly corrosive, and has a high health and safety risk.

What is claimed is:

1. A method of application of sterilization solution comprising: delivering said sterilization solution in the form of an atomized spray.

2. A method of application of sterilization solution according to claim 1 including delivering said sterilization solution in the form of an atomized fog.

3. A method of application of sterilization solution according to claim 1 wherein said sterilization solution comprises a water solution.

4. A method of application of sterilization solution according to claim 3 wherein said water solution contains 3% to 29% hydrogen peroxide.

5. A method of application of sterilization solution according to claim 3 wherein said water solution contains 3% to 10% hydrogen peroxide.

6. A method of application of sterilization solution according to claim 3 wherein said water solution contains about 3% hydrogen peroxide.

7. A method of application of sterilization solution according to claim 1 including delivering said sterilization in a water solution of 3-10% hydrogen peroxide in the form of an atomized fog.

8. A sterilization solution comprising:
a water solution of 3-29% hydrogen peroxide in the form of an atomized fog.

9. A sterilization solution solution according to claim 8 wherein said water solution contains 3% to 10% hydrogen peroxide.

10. A sterilization solution solution according to claim 8 wherein said water solution contains about 3% hydrogen peroxide.
